(19) United States
(12) Patent Application Publication
(10) Pub. No.: US 2008/0033038 A1
(43) Pub. Date: Feb. 7, 2008

(54) Compositions of polyphenols and methods of use

(76) Inventors: R. Douglas Shytle, Tampa, FL (US); Jun Tan, Tampa, FL (US)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 11/825,346

(22) Filed: Jul. 5, 2007

Related U.S. Application Data
(60) Provisional application No. 60/819,527, filed on Jul. 7, 2006.
(60) Provisional application No. 60/819,248, filed on Jul. 7, 2006.

(51) Int. Cl. A61K 31/352; A61P 11/00; A61P 25/00; A61P 29/00; A61P 31/00; A61P 37/00

(52) U.S. Cl. 514/456

(57) ABSTRACT

The subject invention concerns materials and methods for treating or preventing any condition or disease that is treatable by a green tea polyphenol, or an analog, isomer, metabolite, or prodrug thereof, wherein the polyphenol is administered as an intranasal formulation to a person or animal in need of treatment. In one embodiment, the intranasal polyphenol formulation comprises (−)-epigallocatechin-3-gallate (EGCG) and epicatechin (EC), two polyphenols derived from green tea and other plants and that can be produced synthetically. The subject invention also concerns materials and methods for treating or preventing dementia, such as AIDS-dementia in a person or animal by administering a therapeutically effective amount of a polyphenol, or an analog, isomer, metabolite, or prodrug thereof. In one embodiment, a composition of the invention increases expression or activity of a protein that exhibits α-secretase activity.

### CROSS REFERENCE TO RELATED APPLICATION

This application claims the benefit of U.S. Provisional Application Ser. No. 60/819,527, filed Jul. 7, 2006 and U.S. Provisional Application Ser. No. 60/819,248, filed Jul. 7, 2006, the disclosure of each of which is hereby incorporated by reference in their entirety, including all figures and tables.

### BACKGROUND OF THE INVENTION

Green tea, the beverage made from the unfermented leaves of camellia sinensis, is one of the most ancient and widely consumed beverages in the world. Green tea polyphenols have demonstrated significant antioxidant properties. On the basis of a large body of evidence, it has become clear that compounds from green tea play different roles in antioxidant and other functions.

There is increasing evidence supporting the central role of antioxidant effects in opposing aging-related diseases. Recent studies suggest that green tea may be employed for the prevention and treatment of multiple neurodegenerative diseases including Alzheimer's disease (AD) and other forms of dementia (Okello et al., 2004). However, there are no reports as to the active ingredients in green tea that have beneficial effects on neurodegenerative conditions.

Amyloid precursor protein (APP) proteolysis is the fundamental process for the production of β-amyloid (Aβ) peptides which can be deposited as plaques in brain tissue and which are implicated in Alzheimer's disease (AD) pathology (Golde et al., 2000; Huse and Doms, 2000; Sambamurti et al., 2002; Funamoto et al., 2004). APP proteolytic products arise from the coordinated action of α-, β-, and γ-secretases. In the amyloidogenic pathway, Aβ peptides are produced by the initial action of β-secretase (BACE) cleavage, which creates an Aβ-containing C-terminal fragment (CTF) known as β-CTF or C99 (Sinha and Lieberburg, 1999; Yan et al., 1999). This proteolysis also generates an N-terminal, soluble APP-β (sAPP-β) fragment, which is released extracellularly. Intracellularly, β-CTF is then cleaved by a multi-protein γ-secretase complex that results in generation of the Aβ peptide and a smaller γ-CTF, also known as C57 (De Strooper et al., 1998; Steiner et al., 1999). Conversely, in the nonamyloidogenic pathway, APP is first cleaved at the α-secretase site, which results in the release of N-terminal sAPP-α and the generation of α-CTF or C83 (Hooper and Turner, 2002), events that are indicative of α-secretase activity (Hooper and Turner, 2002). Cleavage within the Aβ domain of APP results in two nonamyloidogenic pieces and thereby prevents Aβ peptide generation from that APP (Lichtenthaler et al., 2004). Because of the limiting amount of APP in the cell and the failure to saturate the BACE pathway during APP overexpression, it is believed that the above-mentioned amyloidogenic and nonamyloidogenic pathways compete for substrate in the process of APP proteolysis (Gandhi et al., 2004). Therefore, it is often inferred that extracellular elevation of sAPP-α (resulting from nonamyloidogenic pathway activation) can be taken as indirect evidence of inhibition of BACE and the associated amyloidogenic pathway. However, because the extracellular secretion of these various fragments can be regulated independently of APP cleavage, it is important to fully characterize the effects of treatment on both pathways concurrently before making inferences about underlying mechanisms (Rossner et al., 2000).

Over the past decade, intense focus has been given to investigating the processes of APP proteolysis and Aβ metabolism as possible targets for AD therapy (Hardy and Selkoe, 2002). Various synthetic and naturally occurring compounds have been analyzed for their efficacy in the modulation of these pathological events. One such naturally occurring compound achieving worldwide popularity for its therapeutic application is green tea. Green tea contains polyphenolic structures categorized as flavonoids, which are believed to be the active components accounting for the therapeutic properties of green tea. One green tea compound, (−) -epigallocatechin-3-gallate (EGCG), has been extensively studied primarily because of its reported anticarcinogenic effects (Lin and Liang, 2000; Moyers and Kumar, 2004). Recently, EGCG has been found to modulate protein kinase C (PKC) activity and to consequently increase secreted levels of sAPP-α (Levites et al., 2002; Levites et al., 2003). Additionally, EGCG has been shown to inhibit various activities of proinflammatory cytokines (Ahmed et al., 2002; Han, 2003; Li et al., 2004). Accordingly, signal transducer and activator of transcription 1 and nuclear factor KB responses are inhibited by EGCG (Han, 2003; Aktas et al., 2004). Elucidation of these molecular actions of EGCG substantiates the compound as a versatile modulator of cellular responses that may contribute to disease pathogenesis.

A number of reports have implicated members of the α-disintegrin-and-metalloprotease (ADAM) family, a family of zinc metalloproteases that includes ADAM9, 10, and 17, as putative α-secretase candidates (Hooper et al., 2002; Allinson et al., 2003; Asai et al., 2003). Lammich and colleagues first described the ability of ADAM10 to act as an α-secretase (Lammich et al., 1999), whereas Buxbaum and co-workers reported that ADAM17 contributes to α-secretase processing of APP (Buxbaum et al., 1998). Others have demonstrated the ability of ADAM9 to promote α-secretase cleavage (Hotoda et al., 2002). However, Asai and colleagues reported that ADAM9, 10, and 17 all have roles in the processing of APP to sAPP-α in vitro (Asai et al., 2003). In cerebrospinal fluid from AD patients, ADAM10 and corresponding sAPP/α-CTFs are decreased (Colciaghi et al., 2002; Colciaghi et al., 2004). Moreover, ADAM10 is also decreased in AD and Down's syndrome brains (Bernstein et al., 2003). A report by Lopez-Perez and colleagues implicates ADAM10 as a contributor to constitutive sAPP-α production, whereas ADAM17 (also known as TNF-α converting enzyme, TACE) is implicated in a regulated mechanism of sAPP-α production (Lopez-Perez et al., 2001). Recently, Postina et al., 2004 showed that activation of α-secretase significantly reduces AD-like pathology in an animal model of AD (Postina et al., 2004).

### BRIEF SUMMARY OF THE INVENTION

The subject invention concerns materials and methods for treating or preventing any condition or disease that is treatable by a composition comprising a polyphenol, or an analog, isomer, metabolite, or prodrug thereof, wherein the polyphenol is administered as an intranasal formulation to a person or animal in need of treatment. In one embodiment, a composition increases expression or activity of a protein that exhibits α-secretase activity. In one embodiment, the intranasal polyphenol formulation comprises (−)-epigallocatechin-3-gallate (EGCG) and/or epicatechin (EC), two polyphenols derived from green tea and other plants and that can be produced synthetically.

The subject invention also concerns materials and methods for treating or preventing dementia, such as Acquired Immune Deficiency Syndrome (AIDS)-Dementia, resulting from infection by an immunodeficiency virus in a person or animal by administering a therapeutically effective amount of a polyphenol, or an analog, isomer, metabolite, or prodrug thereof. In one embodiment, a composition of the invention increases expression or activity of a protein that exhibits α-secretase activity.

### DETAILED DISCLOSURE OF THE INVENTION

The subject invention concerns materials and methods for treating or preventing any disease or condition treatable by a green tea polyphenol, such as a neurodegenerative condition or disease associated with β-amyloid peptide deposition in neural tissue, in a person or animal wherein the method of the invention comprises intranasal administration of a therapeutically effective amount of a composition comprising a polyphenol, or an analog, isomer, metabolite, or prodrug thereof. In one embodiment, the polyphenol useful in the invention increases expression or activity of a protein that exhibits α-secretase activity. In a further embodiment, the protein that exhibits α-secretase activity is ADAM10. Polyphenols contemplated within the scope of the methods of the invention include epigallocatechin-3-gallate (EGCG) and epicatechin (EC).

In one embodiment, the neurodegenerative disease or condition to be treated is Alzheimer's disease. In another embodiment, the disease or condition is an upper respiratory disease, such as that caused by an infection. In another embodiment, the disease or condition is dementia, such as AIDS-dementia. In a further embodiment, the disease or condition is an oncological disorder such as cancer. In a further embodiment, the disease or condition is an infection by virus or bacteria.

In one embodiment of a method of the invention, the polyphenol increases the cleavage activity of the protein having α-secretase activity. In another embodiment, the polyphenol increases the expression of the gene encoding the protein and/or increases the amount of the protein produced or present in a cell. In one embodiment, the polyphenol has been treated to remove compounds that antagonize α-secretase activity, including but not limited to, (−)-gallocatechin (GC) and (−)-catechin (C).

The subject invention also concerns methods for increasing the cleavage activity of α-secretase by intranasal administration to a person or animal of an effective amount of at least one of the active compounds present in or derived from green tea, including (−)-epigallocatechin-3-gallate (EGCG) and epicatechin (EC) as well as their analogs, isomers, prodrugs, metabolites, or salts thereof. Advantageously, increasing α-secretase activity can be useful in preventing or treating a disease characterized by amyloid deposition in a patient. In one embodiment, the amyloid associated disease is Alzheimer's disease. In some instances, the patient may be asymptomatic of an amyloid disease. In some methods, the patient has environmental and/or genetic risk factors that indicate a susceptibility of developing an amyloid disease. In other methods, the patient has no risk factors.

In the methods of the subject invention, α-secretase levels and/or activity can be elevated enough to 1) reduce pathological levels of Aβ production to normal or nonpathological levels and/or 2) to increase sAPPα to levels that are neuroprotective in a mammalian patient.

One aspect of the subject invention is directed to methods for reducing or preventing elevated levels of amyloid peptides. Specifically, the subject methods can be used to reduce β-amyloid (Aβ) generation within a cell in vivo or in vitro. Advantageously, the methods of the subject invention also enhance the cleavage of tumor necrosis factor α-converting enzyme (TACE).

The subject invention also concerns compositions comprising a polyphenol of the invention in a pharmaceutically acceptable carrier or diluent.

The subject invention also concerns intranasal formulations of a polyphenol of the invention for use or administration as a dietary supplement.

The subject invention also concerns compositions for intranasal administration comprising polyphenols that increase expression or levels of a protein having α-secretase activity, such as ADAM10, and agents or compounds that inhibit or decrease expression or levels of protein having β-secretase activity or γ-secretase activity. In one embodiment, the polyphenols are EGCG and/or EC, and analogs, isomers, metabolites, or prodrugs thereof. Preferably, the polyphenols are provided in purified form. More preferably, the polyphenols are purified to a level wherein compounds that antagonize the activity of the polyphenols are removed or decreased to a level wherein they do not antagonize the action of the polyphenols.

The polyphenol compositions can be purified or treated to remove inactive or antagonistic compounds, such as (−)-gallocatechin and/or (−)-catechin. In one embodiment, the agents or compounds comprise nucleic acid that is antisense to nucleic acid encoding a protein with β-secretase activity or γ-secretase activity, and/or comprise a small interfering RNA (siRNA) molecule that interferes with expression of a protein having β-secretase activity or γ-secretase activity.

The extracts, compounds or combination of compounds derived from green tea that are useful in the subject invention are generally prepared by methods known in the art. Tea extracts containing high concentrations of EGCG and other naturally occurring tea-derived polyphenols are commercially available. With regard to chemical synthesis of the compounds, reference is made to Li et al., (2001), which is incorporated in its entirety by reference.

While the invention is described with respect to tea-derived polyphenol compounds or analogs, from this disclosure the skilled artisan will appreciate and envision synthetic routes to obtain and/or prepare the active compounds, including synthetic tea polyphenols and their derivates. The compounds utilized in the subject methods can be derived from green tea or other plant or food products or can be produced synthetically. Analogs of green tea extracts useful in the compositions and methods of the subject application are known in the art and examples are described in U.S. Pat. No. 6,713,506; Lam, W. H. et al., (2004); Smith, D. M. et al., (2004); and Wan, S. B. et al., (2005), all of which are incorporated in their entirety by reference. Analogs useful in increasing α-secretase cleavage activity should not be analogs to gallocatechin and/or (−)catechin, two green the flavonoids that decrease α-secretase activity.

In one embodiment, the methods of the subject invention comprise intranasal administration of pharmaceutical compositions to a patient. The pharmaceutical compositions comprise at least one active ingredient of green tea in one or more pharmaceutically acceptable carriers. Each carrier must be acceptable in the sense of being compatible with the other ingredients of the formulation and not injurious to the patient. One such composition comprises EGCG, EC, or pharmaceutically acceptable salts, or analogs thereof, or a mixture of any of the foregoing in a pharmaceutically acceptable carrier. The composition can be purified or treated to remove inactive or antagonistic compounds, such as (−)-gallocatechin and/or (−)-catechin.

Intranasal formulations can conveniently be provided or presented in unit dosage form and can be prepared by any methods well known in the art of pharmacy. Such methods include the step of bringing into association the active ingredient with the carrier which constitutes one or more accessory ingredients. In one embodiment, the formulations are prepared by uniformly and intimately bringing into association the active ingredient with liquid carriers or finely divided solid carriers or both.

Materials, methods, and devices for intranasal administration of substances and compounds are known in the art and are contemplated for use with the present invention. Examples include, but are not limited to, those described in U.S. Pat. Nos. 7,204,822; 6,948,492; 6,815,424; 6,398,774; 5,948,749; 5,804,212; 5,707,644; 5,756,104; 5,112,804; 7,214,209; 7,112,332; 6,972,300; and 6,855,332.

Formulations suitable for intranasal administration, wherein the carrier is a solid, include a coarse powder having a particle size, for example, in the range of about 20 to about 500 microns, which is administered in the manner in which snuff is taken, i.e., by rapid inhalation through the nasal passage from a container of the powder held close up to the nose. Suitable formulations wherein the carrier is a liquid for administration by nebulizer, include aqueous or oily solutions of the agent. Formulations preferably comprise compounds that facilitate absorption of the active ingredients through the skin and into the bloodstream.

Preferred unit dosage formulations are those containing a daily dose or unit, daily subdose, as herein above-recited, or an appropriate fraction thereof, of an agent. It should be understood that in addition to the ingredients particularly mentioned above, the formulations useful in the present invention can include other agents conventional in the art regarding the type of formulation in question.

Therapeutic amounts can be empirically determined and will vary with the pathology being treated, the subject being treated, and the efficacy and toxicity of the agent. Similarly, suitable dosage formulations and methods of administering the agents can be readily determined by those of skill in the art.

In addition to a compound of the subject invention, the pharmaceutical compositions can also contain other pharmaceutically active compounds or a plurality of compounds of the invention.

Intranasal formulations of the invention can be administered simultaneously or sequentially with other drugs or biologically active agents. Examples include, but are not limited to, antioxidants, free radical scavenging agents, peptides, growth factors, antibiotics, bacteriostatic agents, immunosuppressives, anticoagulants, buffering agents, anti-inflammatory agents, anti-pyretics, time-release binders, anesthetics, steroids and corticosteroids. In one specific embodiment, the compositions or formulations of the invention are administered simultaneously or sequentially with galantamine, deprenyl, cdp choline, folate, Vitamin B12, Vitamin B6, piracetam, vinpocetine, idebenone, pyritinol, memantine, or a combination of any of the forgoing.

Methods of the invention can also be used to treat or prevent inflammatory or auto-immune diseases of the peripheral nervous system e.g., rheumatoid arthritis, autonomic neuropathy, brachial plexus injuries, cervical radiculopathy, chronic inflammatory demyelinary polyneuropathy, diabetic neuropathies, dysautonomia, erb-duchenne palsy, dejerine-klumke palsy, glossopharyngeal neuralgia, hereditary neuropathies, Isaac's syndrome, and postherpetic neuralgia, or any disease characterized by up-regulated TACE activity.

The subject invention also concerns materials and methods for treating or preventing dementia, such as AIDS-dementia, in a person or animal by administering a therapeutically effective amount of a composition comprising a polyphenol, or an analog, isomer, metabolite, or prodrug thereof. In a preferred embodiment, a composition of the invention is administered to a person or animal via an intranasal route and the composition is formulated for intranasal administration. In one embodiment, a composition increases expression or activity of a protein that exhibits α-secretase activity. In one embodiment, the protein that exhibits α-secretase activity is ADAM10. Polyphenols contemplated within the scope of the methods of the invention include (−)-epigallocatechin-3-gallate (EGCG) and epicatechin (EC). In one embodiment, the person or animal is infected with HIV, SIV, or FIV.

The subject invention also concerns methods for treating dementia, such as AIDS-dementia, by increasing the cleavage activity of α-secretase by administering to a person or animal an effective amount of at least one of the active compounds present in or derived from green tea, including but not limited to (−)-epigallocatechin-3-gallate (EGCG) and epicatechin (EC) as well as their analogs, isomers, prodrugs, metabolites, or salts thereof. Advantageously, increasing α-secretase activity can be useful in preventing or treating a disease or condition that is associated with or characterized by amyloid deposition in a patient.

Each green tea derived polyphenol administered in the methods of the subject invention may also be administered as a drinkable tea. The tea, or a polyphenol composition derived from green tea, may be purified to remove inactive compounds and/or compounds known to antagonize α-secretase activity including, for example, (−)-gallocatechin (GC) and (−)-catechin (C).

The methods of the subject invention may also be practiced by administering pharmaceutical compositions to a patient. The pharmaceutical compositions comprise at least one active ingredient in one or more pharmaceutically acceptable carriers. Each carrier must be acceptable in the sense of being compatible with the other ingredients of the formulation and not injurious to the patient. One such composition comprises EGCG, EC, or pharmaceutically acceptable salts, isomers, prodrugs, metabolites, or analogs thereof, or a mixture of any of the foregoing in a pharmaceutically acceptable carrier.

Formulations include those suitable for oral, rectal, intranasal, topical (including transdermal, buccal and sublingual), vaginal, parental (including subcutaneous, intramuscular, intravenous and intradermal) and pulmonary administration. The formulations can conveniently be presented in unit dosage form and can be prepared by any methods well known in the art of pharmacy. Such methods include the step of bringing into association the active ingredient with the carrier which constitutes one or more accessory ingredients. In general, the formulations are prepared by uniformly and intimately bringing into association the active ingredient with a suitable carrier, such as liquid carriers or finely divided solid carriers or both, and then if necessary shaping the product. Formulations of the subject invention suitable for oral administration can be presented as discrete units such as capsules, cachets or tablets, each containing a predetermined amount of the active ingredient; or as an oil-in-water liquid emulsion, water-in-oil liquid emulsion or as a supplement within an aqueous solution, for example, a tea. The active ingredient can also be presented as bolus, electuary, or paste.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; mouthwashes comprising the active ingredient in a suitable liquid carrier; and chocolate comprising the active ingredients.

Pharmaceutical compositions for topical administration according to the subject invention can be formulated as an ointment, cream, suspension, lotion, powder, solution, paste, gel, spray, aerosol or oil. Alternatively, a formulation can comprise a patch or a dressing such as a bandage or adhesive plaster impregnated with active ingredients, and optionally one or more excipients or diluents. Topical formulations preferably comprise compounds that facilitate absorption of the active ingredients through the skin and into the bloodstream.

Formulations suitable for intranasal administration, wherein the carrier is a solid, include a coarse powder having a particle size, for example, in the range of about 20 to about 500 microns, which is administered in the manner in which snuff is taken, i.e., by rapid inhalation through the nasal passage from a container of the powder held close up to the nose. Suitable formulations wherein the carrier is a liquid for administration by nebulizer, include aqueous or oily solutions of the agent. Formulations preferably comprise compounds that facilitate absorption of the active ingredients through the skin and into the bloodstream.

Formulations suitable for parenteral administration include aqueous and non-aqueous isotonic sterile injection solutions which can contain antioxidants, buffers, bacteriostats and solutes which render the formulation isotonic with the blood of the intended recipient; and aqueous and non-aqueous sterile suspensions which can include suspending agents and thickening agents, and liposomes or other microparticulate systems which are designed to target the compound to blood components or one or more organs. The formulations can be presented in unit-dose or multi-dose or multi-dose sealed containers, such as for example, ampoules and vials, and can be stored in a freeze-dried (lyophilized) condition requiring only the addition of the sterile liquid carrier, for example, water for injections, immediately prior to use. Extemporaneous injection solutions and suspensions can be prepared from sterile powders, granules and tablets of the kind previously described.

In one embodiment, unit dosage formulations contain a daily dose or unit, daily subdose, as herein above-recited, or an appropriate fraction thereof, of an agent. It should be understood that in addition to the ingredients particularly mentioned above, the formulations useful in the present invention can include other agents conventional in the art regarding the type of formulation in question. For example, formulations suitable for oral administration can include such further agents as sweeteners, thickeners, and flavoring agents. It also is intended that the agents, compositions, and methods of this invention be combined with other suitable compositions and therapies.

Various delivery systems are known in the art and can be used to administer a therapeutic agent or composition of the invention, e.g., encapsulation in liposomes, microparticles, microcapsules, receptor-mediated endocytosis and the like. Methods of delivery include, but are not limited to, intra-arterial, intramuscular, intravenous, intranasal, and oral routes. In a specific embodiment, the pharmaceutical compositions of the invention can be administered locally to the area in need of treatment; such local administration can be achieved, for example, by local infusion during surgery, by injection, or by means of a catheter.

The pharmaceutical compositions can be administered by any of a variety of routes, such as orally, intranasally, parenterally or by inhalation therapy, and can be provided in the form of tablets, lozenges, granules, capsules, pills, ampoule, suppositories or aerosol form. The pharmaceutical compositions can also be provided in the form of suspensions, solutions, and emulsions of the active ingredient in aqueous or nonaqueous diluents, syrups, granulates or powders.

In one embodiment, a compound or composition of the invention is administered in a manner so as to achieve peak concentrations of the active compound at sites of the disease. Peak concentrations at disease sites can be achieved, for example, by intravenously injecting of the agent, optionally in saline, or orally administering, for example, a tablet, capsule or syrup containing the active ingredient.

The compositions can be administered simultaneously or sequentially with other drugs or biologically active agents. Examples include, but are not limited to, antioxidants, free radical scavenging agents, peptides, growth factors, antibiotics, bacteriostatic agents, immunosuppressives, anticoagulants, buffering agents, anti-inflammatory agents, anti-pyretics, time-release binders, anesthetics, steroids and corticosteroids. In one specific embodiment, the compositions are administered simultaneously or sequentially with galantamine, deprenyl, cdp choline, folate, Vitamin B12, Vitamin B6, piracetam, vinpocetine, idebenone, pyritinol, memantine, or a combination of any of the foregoing.

All of the methods of the present invention optionally comprise identifying a person or animal in need of treatment.

For the purpose of this invention the following terms are defined below:

It will be understood that a specific “effective amount” for any particular in vivo or in vitro application will depend upon a variety of factors including the activity of the specific compound employed, the age, body weight, general health, sex, and/or diet of the individual, time of administration, route of administration, rate of excretion, drug combination and the severity of the particular disease undergoing prevention or therapy. For example, the “effective amount” may be the amount of compound of the invention necessary to achieve increased α-secretase activity in vivo or in vitro. The “effective amount” may be the amount of compound of the invention necessary to enhance the cleavage of tumor necrosis factor α-converting enzyme.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include citric acid, lactic acid, tartaric acid, fatty acids, and the like. Salts may also be formed with bases. Such salts include salts derived from inorganic or organic bases, for example alkali metal salts such as magnesium or calcium salts, and organic amine salts such as morpholine, piperidine, dimethylamine or diethylamine salts.

As used herein, the term “pharmaceutically acceptable carrier” includes any and all solvents (such as phosphate buffered saline buffers, water, saline and the like), dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in therapeutic compositions is contemplated. Supplementary active ingredients can also be incorporated into the compositions. The pharmaceutical compositions of the subject invention can be formulated according to known methods for preparing pharmaceutically useful compositions. Formulations are described in a number of sources which are well known and readily available to those skilled in the art. For example, Remington's Pharmaceutical Science (Martin, E. W., 1995) describes formulations which can be used in connection with the subject invention.

Reference herein to increased expression or activity of an α-secretase enzyme refers to any form of increase in expression or activity, including, but not limited to an increase in transcription of a gene encoding an enzyme with α-secretase activity; an increase in half-life of an RNA molecule encoding the enzyme; an increase in translation of the RNA into a protein having α-secretase activity; an increase in the half-life of the protein having α-secretase activity, and any other means that results in an increase in the amount of protein produced or present in the cell; or an increase in the enzymatic activity of the protein having α-secretase activity.

As used herein, the terms “individual” and “patient” are used interchangeably to refer to any vertebrate, mammalian species, such as humans and animals. Mammalian species which benefit from the disclosed methods of treatment include, and are not limited to, apes, chimpanzees, orangutans, humans, monkeys; domesticated animals (e.g., pets) such as dogs, cats, guinea pigs, hamsters, Vietnamese pot-bellied pigs, rabbits, and ferrets; domesticated farm animals such as cows, buffalo, bison, horses, donkey, swine, sheep, and goats; exotic animals typically found in zoos, such as bear, lions, tigers, panthers, elephants, hippopotamus, rhinoceros, giraffes, antelopes, sloth, gazelles, zebras, wildebeests, prairie dogs, koala bears, kangaroo, opossums, raccoons, pandas, hyena, seals, sea lions, elephant seals, otters, porpoises, dolphins, and whales. Human or non-human animal patients can range in age from neonates to elderly.

The term “administering” and “administration” is intended to mean a mode of delivery including, without limitation, oral, rectal, parenteral, subcutaneous, intravenous, intramuscular, intraperitoneal, intraarterial, transdermally or via a mucus membrane. One skilled in the art recognizes that suitable forms of oral formulation include, but are not limited to, a tablet, a pill, a capsule, a lozenge, a powder, a sustained release tablet, a liquid, a liquid suspension, a gel, a syrup, a slurry, a suspension, and the like. For example, a daily dosage can be divided into one, two or more doses in a suitable form to be administered at one, two or more times throughout a time period.

The term “therapeutically effective” is intended to mean an amount of a compound sufficient to substantially improve some symptom associated with a disease or a medical condition. For example, in the treatment of cancer, a compound which decreases, prevents, delays, suppresses, or arrests any symptom of the disease would be therapeutically effective. A therapeutically effective amount of a compound is not required to cure a disease but will provide a treatment for a disease such that the onset of the disease is delayed, hindered, or prevented, or the disease symptoms are ameliorated, or the term of the disease is changed or, for example, is less severe or recovery is accelerated in an individual.

The term “analog” is intended to mean a compound that is similar or comparable, but not identical, to a reference compound, i.e. a compound similar in function and appearance, but not in structure or origin to the reference compound. For example, the reference compound can be a reference green tea polyphenol and an analog is a substance possessing a chemical structure or chemical properties similar to those of the reference green tea polyphenol. As used herein, an analog is a chemical compound that may be structurally similar to another but differs in composition (as in the replacement of one atom by an atom of a different element or in the presence of a particular functional group). An analog may be extracted from a natural source or be prepared using synthetic methods.

The term “prodrug” is intended to mean a compound which is administered in an inactive (or less active) form that is metabolized in vivo into an active (or more active) form.

The terms “treatment”, “treating” and the like are intended to mean obtaining a desired pharmacologic and/or physiologic effect, e.g., increasing activity of α-secretase. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease in a mammal, particularly a human, and includes: (a) preventing a disease or condition (e.g., preventing amyloid disease) from occurring in an individual who may be predisposed to the disease but has not yet been diagnosed as having it; (b) inhibiting the disease, (e.g., arresting its development); or (c) relieving the disease (e.g., reducing symptoms associated with the disease).

As used in this specification, the singular forms “a”, “an”, and “the” include plural references unless the context clearly dictates otherwise.

The terms “comprising”, “consisting of”, and “consisting essentially of” are defined according to their standard meaning and may be substituted for one another throughout the subject application in order to attach the specific meaning associated with each term.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

U.S. Pat. No. 6,713,506
U.S. Pat. No. 7,204,822
U.S. Pat. No. 6,948,492
U.S. Pat. No. 6,815,424
U.S. Pat. No. 6,398,774
U.S. Pat. No. 5,948,749
U.S. Pat. No. 5,804,212
U.S. Pat. No. 5,707,644
U.S. Pat. No. 5,756,104
U.S. Pat. No. 5,112,804
U.S. Pat. No. 7,214,209
U.S. Pat. No. 7,112,332
U.S. Pat. No. 6,972,300
U.S. Pat. No. 6,855,332
Ahmed S. et al., (2002) “Green tea polyphenol epigallocatechin-3-gallate inhibits the IL-1 beta-induced activity and expression of cyclooxygenase-2 and nitric oxide synthase-2 in human chondrocytes” Free Radic Bio Med 33:1097-1105.
Aktas O. et al., (2004) “Green tea epigallocatechin-3-gallate mediates T cellular NF-kappaB inhibition and exerts neuroprotection in autoimmune encephalomyelitis” J. Immunol 173:5794-5800.
Allinson, T. M. et al., (2003) J. Neurosci. Res. 74:342-352.
Asai, M. et al., (2003) Biochem. Biophys. Res. Commun. 301:231-235.
Bernstein, H. G. et al., (2003) J. Neurocytol. 32:153-160.
Buxbaum, J. D. et al., (1998) J. Biol. Chem. 273:27765-27767.
Colciaghi, F. et al., (2002) Mol. Med. 8:67-74.
Colciaghi, F. et al., (2004) Neurology 62:498-501.
De Strooper B. et al., (1998) “Deficiency of presenilin-1 inhibits the normal cleavage of amyloid precursor protein” Nature 391:387-390.
Funamoto S. et al., (2004) “Truncated carboxyl-terminal fragments of beta-amyloid precursor protein are processed to amyloid beta-proteins 40 and 42” Biochemistry 43:13532-13540.
Gandhi S. et al., (2004) “Amyloid precursor protein compartmentalization restricts beta-amyloid production: therapeutic targets based on BACE compartmentalization” J. Mol Neurosci 24:137-143.
Golde T. E. et al., (2000) “Biochemical detection of Abeta isoforms: implications for pathogenesis, diagnosis, and treatment of Alzheimer's disease” Biochim Biophys Acta 1502:172-187.
Han M. K. (2003) “Epigallocatechin gallate, a constituent of green tea, suppresses cytokine-induced pancreatic beta-cell damage” Exp Mol Med 35:136-139.
Hardy J. and Selkoe D. J. (2002) “The amyloid hypothesis of Alzheimer's disease: progress and problems on the road to therapeutics” Science 297:353-356.
Hooper N. M. and Turner A. J. (2002) “The search for alpha-secretase and its potential as a therapeutic approach to Alzheimer's disease” Curr Med Chem 9:1107-1119.
Hotoda, N. et al., (2002) Biochem. Biophys. Res. Commun. 293:800-805.
Hsiao K, et al., (1996) “Correlative memory deficits, Abeta elevation, and amyloid plaques in transgenic mice” Science 274:99-102.
Huse J. T. et al., (2000) “Closing in on the amyloid cascade: recent insights into the cell biology of Alzheimer's disease” Mol Neurobiol 22:81-98.
Lam, W. H. et al., (2004) “A Potential Prodrug for a Green Tea Polyphenol Proteasome Inhibitor: Evaluation of the Peracetate Ester of (−)-Epigallocatechin Gallate [(−)-EGCG]” Bioorganic & Medicinal Chemistry, 12:5587-5593.
Lammich, S. et al. (1999) Proc. Natl. Acad. Sci. U.S.A. 96:3922-3927.
Li et al., (2001) “Enantioselective Synthesis of Epigallocatechin-3-gallate (EGCG), the Active Polyphenol Component from Green Tea” Organic Letters, 3:739-41.
Li, R. et al., (2004) “(−)-Epigallocatechin gallate inhibits lipopolysaccharide-induced microglial activation and protects against inflammation-mediated dopaminergic neuronal injury” J Neurosci Res., 78:723-31.
Lichtenthaler et al., (2004) “Amyloid at the cutting edge: activation of α-secretase prevents amyloidogenesis in an Alzheimer disease mouse model” J. Clin. Invest., 113:1384-87.
Lin J. K. and Liang Y. C. (2000) “Cancer chemoprevention by tea polyphenols” Proc Natl Sci Counc Repub China 1: 1-13.
Levites Y. et al., (2002) “Involvement of protein kinase C activation and cell survival/cell cycle genes in green tea polyphenol (−)-epigallocatechin 3-gallate neuroprotective action” J Biol Chem 277:30574-30580.
Levites Y. et al., (2003) “Neuroprotection and neurorescue against Abeta toxicity and PKC-dependent release of nonamyloidogenic soluble precursor protein by green tea polyphenol (−)-epigallocatechin-3-gallate” FASEB J 17:952-954.
Lopez-Perez, E. et al., (2001) J. Neurochem. 76:1532-1539.
Moyers S. B. and Kumar N. B. (2004) “Green tea polyphenols and cancer chemoprevention: multiple mechanisms and endpoints for phase II trials” Nutr Rev 62:204-211.
Okello et al., (2004) “In vitro Anti-β-secretase and Duel Anti-cholinesterase Activities of Camellia sinensis L. (tea) Relevant to Treatment of Dementia” Phytother. Res., 18: 624-27.
Postina, R. et al., (2004) J. Clin. Invest 113:1456-1464.
Remington's Pharmaceutical Science (Martin E W (1995) Easton Pa., Mack Publishing Company, 19th ed.
Rossner S. et al., (2000) “Constitutive overactivation of protein kinase C in guinea pig brain increases alpha-secretory APP processing without decreasing beta-amyloid generation” Eur J Neurosci 12:3191-3200.
Sambamurti K. et al., (2002) “Advances in the cellular and molecular biology of the beta-amyloid protein in Alzheimer's disease” Neuromolecular Med 1: 1-31.
Sinha S. and Lieberburg I. (1999) “Cellular mechanisms of beta-amyloid production and secretion” Proc Natl Acad Sci USA 96:11049-11053.
Smith, D. M. et al., (2004) “Docking Studies and Model Development of Tea Polyphenol Proteasome Inhibitors: Applications to Rational Drug Design” PROTEINS. Structure, Function, and Bioinformatics, 54:58-70.
Steiner H. et al., (1999) “A loss of function mutation of presenilin-2 interferes with amyloid beta-peptide production and notch signaling” J. Biol Chem 274:28669-28673.
Wan, S. B. et al., (2005) “Structure-activity Study of Epi-gallocatechin Gallate (EGCG) Analogs as Proteasome Inhibitors” Bioorganic & Medicinal Chemistry, 13:2177-2185.
Yan R. et al., (1999) “Membrane-anchored aspartyl protease with Alzheimer's disease beta-secretase activity” Nature 402:533-537.

We claim:

1. A method for treating or preventing a condition or disease that is treatable by a composition comprising a green tea polyphenol, or an analog, isomer, metabolite, or a prodrug thereof, wherein said method comprises administering an effective amount of said polyphenol as an intranasal formulation to a person or animal in need of treatment.

2. The method according to claim 1, wherein said green tea polyphenol increases expression or activity of a protein that exhibits α-secretase activity.

3. The method according to claim 1, wherein said green tea polyphenol formulation comprises (−)-epigallocatechin-3-gallate (EGCG) and/or epicatechin (EC).

4. The method according to claim 1, wherein said condition or disease is a neurodegenerative condition or disease associated with β-amyloid peptide deposition in neural tissue.

5. The method according to claim 4, wherein said neurodegenerative condition or disease is Alzheimer's disease.

6. The method according to claim 1, wherein said condition or disease is an upper respiratory disease.

7. The method according to claim 1, wherein the said condition or disease is dementia.

8. The method according to claim 1, wherein said condition or disease is an oncological disorder.

9. The method according to claim 1, wherein said condition or disease is an infection caused by a virus, fungus, or bacteria.

10. The method according to claim 1, wherein said polyphenol increases the expression of a gene encoding a protein having α-secretase activity and/or increases the amount of a protein having α-secretase activity produced or present in a cell.

11. A composition comprising a green tea polyphenol, wherein said composition is formulated for intranasal administration.

12. The composition according to claim 11, wherein said composition comprises EGCG and/or EC, or an analog, isomer, prodrug, metabolite, or salt thereof.

13. The composition according to claim 11, wherein said composition comprises a carrier.

14. The composition according to claim 13, wherein said carrier comprises particles, wherein said particles are from about 20 microns to about 500 microns in diameter.

15. The composition according to claim 13, wherein said carrier is a liquid.

16. The composition according to claim 11, wherein said composition comprises one or more compounds that facilitate absorption of said green tea polyphenol through a person or animal's skin and into the bloodstream.

17. A method for treating or preventing an inflammatory or autoimmune condition or disease, wherein said method comprises administering an effective amount of a composition comprising a green tea polyphenol, or an analog, isomer, metabolite, prodrug, or salt thereof to a person or animal having or predisposed to having said inflammatory or autoimmune condition or disease.

18. The method according to claim 17, wherein said inflammatory or autoimmune condition or disease is selected from the group consisting of rheumatoid arthritis, autonomic neuropathy, brachial plexus injuries, cervical radiculopathy, chronic inflammatory demyelinary polyneuropathy, diabetic neuropathies, dysautonomia, erb-duchenne palsy, dejerine-klumke palsy, glossopharyngeal neuralgia, hereditary neuropathies, Isaac's syndrome, postherpetic neuralgia, and any disease characterized by up-regulated TACE activity.

19. A method for treating or preventing dementia in a person or animal by administering an effective amount of a composition comprising a green tea polyphenol, or an analog, isomer, metabolite, prodrug, or salt thereof, to the person or animal.

20. The method according to claim 19, wherein said composition is administered to said person or animal via an intranasal route.

21. The method according to claim 19, wherein said composition increases expression or activity of a protein that exhibits α-secretase activity.

22. The method according to claim 21, wherein said protein that exhibits α-secretase activity is ADAM10.

23. The method according to claim 19, wherein said green tea polyphenol comprises EGCG and/or EC, or an analog, isomer, prodrug, metabolite, or salt thereof.

24. The method according to claim 19, wherein said person or animal is infected with HIV, SIV, or FIV.

25. The method according to claim 19, wherein said green tea polyphenol is purified to remove one or more compounds that antagonize α-secretase activity.

26. The method according to claim 25, wherein said one or more compounds that antagonize α-secretase activity are (−)-gallocatechin (GC) and (−)-catechin (C).

27. The method according to claim 19, wherein the dementia is AIDS-dementia.

28. A method for treating or preventing dementia in a person or animal by increasing the cleavage activity of an α-secretase protein or for treating or preventing an elevated level of amyloid peptide in a person or animal, said method comprising administering to a person or animal an effective amount of at least one of the active compounds present in or derived from green tea.

29. The method according to claim 28, wherein said active compound derived from green tea is EGCG or EC, or an analog, isomer, prodrug, metabolite, or salt thereof.

30. The method according to claim 28, wherein said active compound derived from green tea is administered as a drinkable tea.

31. The method according to claim 28, wherein said active compound derived from green tea is purified to remove one or more compounds that antagonize α-secretase activity.

32. The method according to claim 31, wherein said one or more compounds that antagonize α-secretase activity are (−)-gallocatechin (GC) and (−)-catechin (C).

33. The method according to claim 28, wherein said active compound derived from green tea is administered as a pharmaceutical composition.

34. The method according to claim 28, wherein the dementia is AIDS-dementia.

35. The method according to claim 28, wherein said amyloid peptide is β-amyloid peptide.
